(19) United States
(12) Patent Application Publication
(10) Pub. No.: US 2015/0168436 A1
(43) Pub. Date: Jun. 18, 2015

(54) ANALYSIS APPARATUS AND METHOD FOR AUTOMATED DETERMINATION OF A MEASUREMENT VARIABLE SELECTED FROM SEVERAL MEASUREMENT PARAMETERS

(71) Applicant: Endress+Hauser Conducta GmbH + Co. KG, Weil am Rhein (DE)

(72) Inventors: Ulrich Kathe, Ludwigsburg (DE); Ralf Steuerwald, Eberdingen-Hochdorf (DE)

(21) Appl. No.: 14/566,092

(22) Filed: Dec. 10, 2014

(30) Foreign Application Priority Data
Dec. 13, 2013 (DE) DE102013114011.4

(51) Int. Cl. G01N 35/10; G01N 33/18

(52) U.S. Cl. CPC G01N 35/1097; G01N 33/182

(57) ABSTRACT

An analyzer automatically determines the measurement variable for a liquid sample selected from a plurality of measurement parameters. An electronic control is configured to control the analyzer based on a selection signal representing the measurement variable selected from the plurality of measurement parameters, in particular, the conveying and metering devices and the photometric measurement device. The controller controls the analyzer in order to determine the selected measurement variable, and/or, based on the selection signal, the control unit is configured to determine a measurement value for the selected measurement variable by using the measurement signal provided by the photometric measuring device.

### REFERENCE TO RELATED APPLICATION

This application claims priority to German patent application No. DE102013114011.4, filed Dec. 13, 2013, the entire content of which is incorporated herein by reference.

### FIELD OF THE INVENTION

This invention relates to an analyzer for automatically determining the measured value of a liquid sample selected from several measurement parameters.

### BACKGROUND OF THE INVENTION

Analyzers are used in process measurement technology and industrial metrology. For example, analyzers can be used to monitor and optimize cleaning performance of a wastewater treatment plant. Furthermore, analyzers can be used for monitoring drinking water or for monitoring the quality of food. Examples of measured variables that analyzers determine and monitor are concentrations of specific substances in liquid, e.g. a concentration of ions, such as ammonium, phosphate or nitrate, the amount of biological or biochemical compounds, e.g. hormones, or micro-organism content. Other measured variables, which are determined by analyzers in process measurement technology, particularly in the area of monitoring water, are the total carbon content (TOC) or chemical oxygen demand (COD), which, as a sum parameter, are dependent on the concentration level of several carbonaceous and/or oxygen-consuming substances in the liquid to be monitored.

Often in analyzers, sample liquid to be analyzed is mixed with one or more reagents so that a chemical reaction occurs, which is then detected using physical methods, e.g. optical measurements. For example, the chemical reaction can color the liquid sample or make it change color entirely, which is photometrically detected.

Automatic analyzers are known from prior art. In DE 10 2009 029 305 A1, an analyzer is described that automatically determines a measurement variable in a fluid sample, for example, having one or more liquid containers for one or more fluids, e.g. reagents, a measuring cell for recording a reaction mixture produced by mixing the liquid sample with one or more reagents, and a measuring apparatus for providing one or more measurement signals correlated with the measurement variable. The analysis unit comprises an electronic unit which comprises a control unit for controlling the analyzer and for determining the measured parameter based on the measurement signals provided by the measuring device. The analysis device further comprises a process engineering unit controlled by the control unit that includes a conveying and metering device for conveying and metering the liquid sample and liquids from the liquid container into the measuring cell. The analyzer has at least one replaceable cartridge into which the liquid container and/or at least parts of the process engineering unit are integrated. One advantage of this configuration is that the liquid container or the wear parts on the process engineering unit can be configured in the cassette as hoses or wear parts for the conveying and metering device, which the user must replace at intervals. To provide new liquids or replace wear parts, the user simply has to replace the “used” cassette containing the liquid containers whose fluids need to be changed or containing the wear parts with a new cartridge cassette.

DE 10 2011 075 762 A1 describes an analyzer for automated determination of a measurement variable in a test liquid. The analyzer comprises a measuring cell for receiving the liquid to be measured, and a sensor for detecting a measured value correlated with the measurement variable. The measured value relates to the measuring fluid collected in the measuring cell. The analyzer also comprises a control unit which is configured to determine the measurement variable on the basis of the measured value detected by the sensor. For the purpose of conveying and metering the liquid sample and the liquid sample to be added to the reagents in the measurement cell, the analyzing apparatus comprises a system of fluid lines, a first liquid tank which is connected to the measuring cell by means of at least one initial valve via a lockable initial fluid path in a system of fluid lines, which is connected to a measuring cell by means of a second lockable fluids container, by means of at least one second valve in a system of fluids lines.

The first fluid container is associated with a first pump for transporting fluid along the first fluid path, and wherein a second pump, distinct from the first pump, is associated with the second fluid container, and this second pump is used to transport fluids along the second fluids path. The analyzer comprises a central valve switching device for actuating the first and second valves. The analyzer can be regulated by the control unit.

Often such devices are configured as automatic analyzer cabinet units, wherein an analyzer is configured for determining a single measurement parameter that is correlated with the concentration of one or more substances in the liquid sample. This implies that a user, e.g. a plant operator, must acquire and operate his own analyzer to measure each different measurement parameter. Neither a temporary switchover nor a permanent conversion from one measurement parameter to another measuring parameter is provided or available for the analytical devices known from the prior art.

Since the analyzers are relatively complex in their construction, and correspondingly expensive to purchase and maintain, it would be desirable to be able to switch the analyzers over as needed for determining a different measurement, instead of using the same measurement parameter that was used previously. This would avoid having to purchase, operate in parallel and maintain several analyzers for two or more measuring parameters that have to be determined using alternative methods. It would also be desirable to permanently reequip the analyzers in order to use them more flexibly.

### SUMMARY OF THE INVENTION

The present invention improves upon the existing art by providing an analyzer that the user can convert in a simple manner and/or reequip. The preferred embodiment involves an analyzer for automatically determining the measured value of a liquid sample selected from several measurement parameters. The analyzer comprises:
At least one liquid container for a reagent that is to be added to a liquid sample;
A measuring cell for receiving a reaction mixture formed by the liquid sample mixed with the reagent and a photometric measuring system, which is configured to provide a measurement signal correlated with the measurement variable for the reaction mixture received in the measuring cell;
A conveying and metering device for conveying and metering the liquid sample and reagent into the measuring cell; and
An electronic control unit for controlling the analyzer;

In accordance with the invention, the control unit is configured to control the analyzer, specifically the conveying and metering device and the photometric measuring system, based on a selection signal representing the measurement variable selected from the plurality of measurement parameters in order to determine the selected measurement variable. In addition, or alternatively, the control unit is configured to determine the selected measurement variable from the measurement signal provided by the photometric measuring device based on the selection signal.

The selection signal in particular preferably includes a signal that can be processed by the control unit, a value that can be processed by the control unit, an input that can be processed by the control unit, or a command that can be processed by the control unit whereby the control unit is capable of identifying the selected measurement variable and the analyzer, in particular, the conveyor and metering device and the photometric measurement device, in order to control determining the selected measurement variable represented by the signal, the value, the input or the command, and/or to determine the measuring signal provided by the photometric measuring device, according to the identified selected measured variable.

The analyzer can be used for determining various alternative metrics because the control unit is configured on the basis of a selection signal to control the analyzer regarding how a measurement variable that is selected from one of several measurement parameters is determined, and optionally, how it is determined (or evaluated) based on the signal provided by the photometric measuring device, while also coordinating with the selected measurement variable. The option of being able to use the same analyzer for determining measurement variables that are selected from several measurement parameters affords the user a great deal of flexibility because the user can use the analyzer according to the user's needs.

If, for example, the user initially used the analyzer for monitoring an ammonium concentration as a first measurement parameter, and at a later point had no need for an analyzer to determine an ammonium concentration as a new measurement parameter, the user can convert the analyzer and continue using it for monitoring the new measuring parameter, such as, for example, a phosphate concentration, in a very simple way by selecting the measurement parameter and by providing reagents required for the new measurement parameter that are contained in the liquid containers.

If the user wants to monitor two or more different measurement parameters that do not necessarily have to be measured simultaneously, the user has the option of temporarily converting the unit from monitoring the one first measurement variable that is selected from several measurement parameters to another, second measurement variable selected from said several measurement parameters, different from the first measurement variable, and then later switching back to the first measurement variable. In this case, unlike in the above-mentioned situation where the device is permanently converted, it is advantageous when the analysis device comprises liquid containers with all the reagents required for determining both measured variables. This avoids having to purchase two independent analyzers, each with its own control unit and conveyor and metering units, and also avoids the greater amount of maintenance required in operating a single analyzer.

“Many” or “several” or a “plurality of” measurement parameters mean two or more parameters—in particular, five or more different measurement parameters.

Each of the many measurement parameters, i.e. each of the measurement parameters that the user has the option of selecting, can correlate with either a single concentration or with several substances contained in the liquid sample. The selectable measuring parameters can, for example, include any of the following: an ammonium concentration, a phosphate concentration, a silica concentration, a hydrazine concentration, a nitrate concentration, a nitrite concentration, an aluminum concentration, an iron concentration, a manganese concentration, a total nitrogen content, water hardness content, total phosphate content, total carbon content, a total content of inorganic carbon (TOC) or chemical oxygen demand (COD).

The control unit can have access to a computer program stored in a memory, whereby at least one measurement-parameter-specific control algorithm for each of the many various measurement parameters is implemented in the computer program. This control algorithm controls the analyzer in order to determine this measurement parameter. In addition, or alternatively, at least one measurement-parameter-specific evaluation algorithm, which is used for determining this measurement parameter, can be used in the computer program for evaluating one of the measurement signals provided by the photometric measuring device. The control unit can be adapted for running the computer program in order to determine a measurement value of the selected measurement variable, and this can be done by using the measurement-parameter-specific control algorithm, which is used for determining the selected measurement variable, and/or by using the measurement-parameter-specific evaluation algorithm, which is used for determining the selected measurement variable.

In particular, the computer program can take the form of several various measurement-parameter-specific computer program modules in the computer's memory, whereby each of these computer program modules is used to determine at least one of the many various measurement parameters. The control unit may include at least one computing device that is equipped with a processor, which is designed to run a basic program. The purpose of the basic program is to install, based on the selection signal representing the selected measuring variable, the computer program module used to determine the selected measuring variable in an internal memory and the processor so that the processor can run the computer program modules that are installed there. Thus, not all of the measurement-parameter-specific computer program modules that are used for determining each possible measurement parameter have to be maintained in the processor's memory. Instead, only those computer program modules are installed in the processor's memory that are required for determining the selected measurement variable, i.e. the modules needed to measure this measurement variable.

Alternatively or additionally, the control unit may be configured to read out the selection signal from a memory that can be connected to and disconnected from the control unit. For example, the memory can be connected to a liquid container, which contains a reagent used for determining the selected measurement variable. The control unit may be adapted to read out from this memory an identifier that simultaneously serves as a selection signal, which represents this reagent or the measurement variable.

In a particularly advantageous embodiment, the control unit may be configured to generate time or event-controlled selection signals for switching the analyzer from the first to a second selected measurement variable and back again—i.e., from the second selected measurement variable to the first selected measurement variable.

The photometric measuring device may comprise a radiation source and a radiation receiver, which outputs a measurement signal that is dependent on the intensity of the radiation that is received. The radiation source and the radiation receiver are configured in such a way that the radiation emitted by the radiation source flows along a measurement path between the source of the radiation and the radiation receiver. This measurement path passes through the reaction mixture contained in the measurement cell. The radiation source can be configured to emit radiation of at least one wavelength that is selected by the control unit.

The radiation source may for example comprise a plurality of light-emitting diodes, which are designed to generate radiation from each of respectively different peak wavelengths. Alternatively or additionally, the radiation source may comprise one or more multi-LEDs. The LEDs and control circuits used to activate them are advantageously arranged on a common circuit board. The control unit may be configured to control the respective light-emitting diode used for determining the selected measurement variable for the emission of measurement radiation. A measurement-parameter-specific control algorithm may comprise the control of the radiation source for emitting radiation of the wavelength appropriate for determining the selected measurement variable. A measurement-parameter-specific evaluation algorithm may include a computation rule for calculating the measured value of the measurement variable as based on the measurement signal supplied by the radiation receiver in dependence on the radiation intensity detected by the radiation receiver for the wavelength appropriate for determining the selected measurement variable.

The analyzer may comprise several liquid containers that contain certain reagents, rinsing fluids or standard solutions for calibrating or adjusting the analyzer. All the liquid containers associated with the analyzer may be connected to the measuring cell via a system of fluid lines. Each of the liquid container connections to the measuring cell can be connected by a particular individual fluid path that extends through the system of fluid conduits and can be locked by means of at least one valve. In one embodiment, an individual pump for transporting fluids along the reaction path can be associated with each fluids path or each liquids container. The conveying and metering device may have a central valve-switching mechanism that can be controlled by the control unit, which can be configured to actuate a multitude, especially all the valves on the analyzer. In addition, at least one other lockable fluids path can be formed by a valve actuatable by a central valve switching mechanism. This fluids path connects a sample model, from which the analyzer removes fluid samples that are to be inspected, with the measurement cell. A measurement-parameter-specific control algorithm can in particular comprise a method of controlling the conveying and metering device, which in particular comprises pumps and the central valve switching mechanism, for which it is ensured that the liquid sample is mixed in a predetermined sequence with reagents from predetermined liquid containers for the formation of the reaction mixture.

The invention also relates to a method for determining a measurement variable selected from one of a plurality of measurement parameters by means of an analyzer, in particular by means of an analyzer according to one of the embodiments described above. The method comprises:
Provision of one or more liquid containers containing reagents to form a reaction mixture with a fluid sample;
Selection of the selected measured quantity from a plurality of measurement parameters, in particular, via an input device for a control unit that is part of the analyzer;
Conveying and metering of a liquid sample from a sample preparation using a conveying and metering device controlled by the electronic control unit that is part of the analyzer;
Conveying and metering of one or more reagents from the one or more fluid reservoirs and forming a reaction mixture from the liquid sample, and the one or more reagents by means of the conveying and metering device, which is controlled by the control unit that is part of the analyzer;
Control of a photometric measuring device by means of a control unit for producing the measurement signal dependent on a selected measurement variable; and
Based on a measurement signal, using a control unit to determine a measurement value for the selected measurement variable.

Based on the selection of the selected measurement quantity, the control unit can run measurement-parameter-specific control algorithms that are used in the conveying and metering device and in the photometric measuring device, or can run the evaluation algorithms used in evaluating the measurement signal supplied by the photometric measuring device in order to determine the selected measurement variable. It can select them from a multitude of measurement-parameter-specific control algorithms or measurement-parameter-specific evaluation algorithms based on a selection signal representing the measurement variable, which algorithms are stored, in particular, in the form of software code in a memory to which the control unit has access, and which serve for determining at least one of said plurality of measurement parameters.

In one embodiment of the method, controlling the photometric measuring device can comprise the following:
Control of a radiation source of a photometric measurement device for generating a reaction mixture by radiant measuring radiation received in a measurement cell of a wavelength based on a selection signal representing the selected measurement quantity; and
Detecting a measurement signal produced by the radiation receiver dependent on an intensity of the measuring radiation impacting a radiation receiver of the photometric measuring device after radiating through the reaction mixture.

The radiation source may comprise in particular a plurality of light-emitting diodes which produce radiation of peak wavelengths which in each case are different from one another; in addition or alternatively, the radiation source may include one or more multi-LEDs. In order to determine the selected measured quantity, the control unit triggers the one light-emitting diode, which is adapted to generate radiation of a suitable wavelength for detecting the selected measured quantity for emitting the measurement radiation.

In a variant of this method, which allows the analyzer to temporarily switch from a first selected from one measurement variable of the plurality of measurement parameters to a different, second measurement variable selected from the plurality of measurement parameters, or to switch back from the second measurement variable back to the first measured variable, one or more of the first liquid containers containing reagents used for determining the first selected measured variable, and one or more of the liquid containers containing reagents used for determining the second selected measurement variable are provided. Based on the selection signal, the control unit then controls the conveying and metering unit in such a way that each of the reagents required for determining the currently selected measure are conveyed in the correct sequence and are added to the liquid sample.

### BRIEF DESCRIPTION OF THE DRAWING

FIG. 1 is a schematic illustration of an analytical device that is used to determine a selected measurement variable from a plurality of possible measurement parameters.

### DETAILED DESCRIPTION OF THE INVENTION

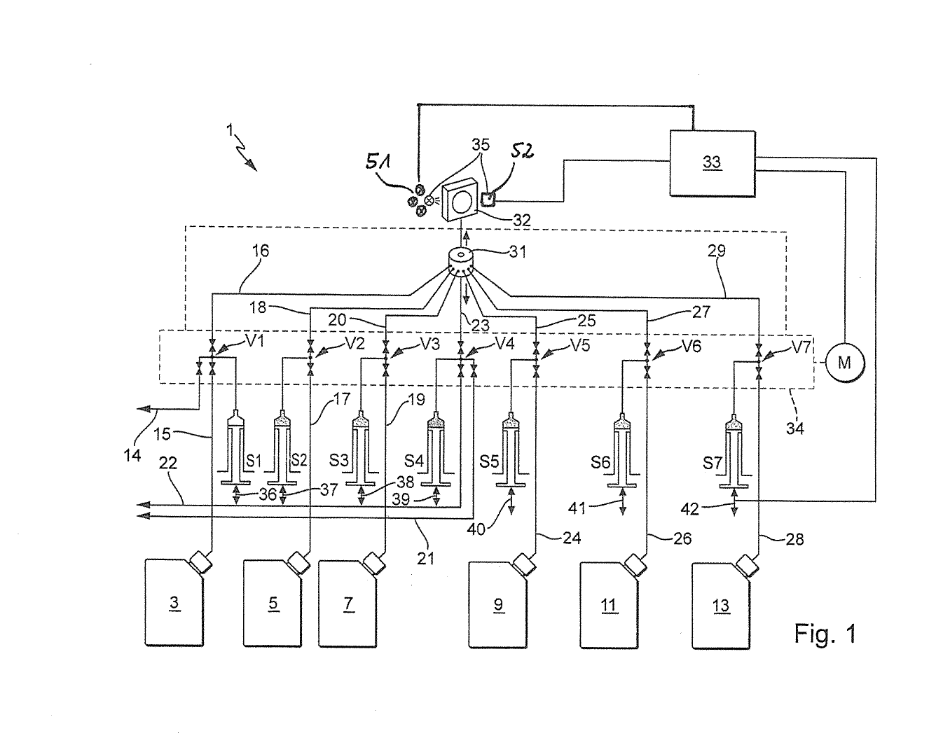

FIG. 1 shows a schematic representation of an analysis device 1 having a plurality of liquid containers 3, 5, 7, 9, 11, 13 and a system of fluid lines 14-29, through which the liquid container(s) 3, 5, 7, 9, 11, 13 are connected via a mixing cell 31 to a measuring cell 32. The fluid paths running from the liquid containers 3, 5, 6, 9, 11, 13 to the measuring cell 32 by means of the system of liquid lines can be blocked in each case by at least one multipath valve V1, V2, V3, V4, V5, V6, V7 and V8. All the valves V1, V2, V3, V4, V5, V6 and V7 are actuated by means of a central valve-switching mechanism 34. The liquid transport along the fluid paths comprised in the system of fluid lines by means of a series of piston pumps S1, S2, S3, S4, S5, S6 and S7. The fluid lines, valves and pumps of the central switching mechanism together form a conveying and metering unit for the analyzer 1.

The valves V1, V2, V3, V4, V5, V6 and V7 may include one or more locking members, which are designed to block or release the associated fluid paths. The central valve switching device 34 may be configured to actuate the locking members electrically, pneumatically or mechanically or in some other way, thus moving the valves V1, V2, V3, V4, V5, V6 and V7 so that they open or block the fluid paths. The blocking elements can for example be designed as movable mounted pins or stamps that can be moved axially by means of associated linear actuators. In this embodiment, the central switching device 34 may include a rotary shaft that has cams or recesses that work together with the valve locking members V1, V2, V3, V4, V5, V6 and V7 to use the valves V1, V2, V3, V4, V5, V6 and V7 to release or block blockable fluid paths. In this embodiment, the shaft can be fitted to the locking members in such a way that during a rotary movement of the shaft around its own axis, the cams or the recesses on the shaft force the blocking members to move. German patent application DE 10 2011 075 762 provides a detailed example of this type of combined valve central valve-switching mechanism.

The analyzer 1 can be completely automated. For this purpose, it has a control unit 33 that functions both as an evaluation unit for determining the measurement variable as well as a control unit for controlling the process performed in the analyzer. The control unit 33 includes a computing device, such as a computer or a transmitter with at least one processor and one or more data storage units that store data and/or programs, which can be read or executed by the computing device. The control unit 33 may also have an input device for inputting commands or parameters by an operator and/or an interface to receive commands, parameters, and other data from a higher-level unit, e.g. from a process control system. Furthermore, the control unit 33 may also include an output device for outputting data, in particular, measurement results or operating information to a user or an interface for outputting data to the parent unit. Additionally or alternatively, the control unit 33 can have an interface via which it can, in particular wirelessly, be connected to an external memory. Via this interface, for example, software updates can be performed, parameters can be read in from the external memory from the control unit 33, or, data generated by the control unit 33 are secured to the external memory. In particular, as is described in further detail below, a selection signal can be read that represents a selected measurement variable to be determined by the analyzer 1. The control unit 33 may also be implemented by linking several, geographically distributed computing devices that are connected to each other for communication purposes.

The control unit 33 is connected to pump drives 36-42 (FIG. 1 only shows the connection with a drive 42 by way of example) of the piston pumps S1, S2, S3, S4, S5, S6 and S7, and connected to a drive M on the central switching station 34, in order to transport liquids to the mixing cell 31, and from there to the measuring cell 32 or out of the measuring cell 32. The control unit 33 is also connected to a photometric measuring device 35, which comprises a radiation source 51 and a radiation receiver 52 in order to control the photometric measuring device 35, and to identify the measurement variable that is to be determined from the measurement signals of the radiation receiver 52. The radiation source 51 comprises in the present example a plurality of light-emitting diodes (LEDs), in particular a plurality of LEDs which each emit different peak wavelength radiation. The radiation receiver 52 may include one or a plurality of photoelectric elements, and in particular may include one or more photodiodes, which are configured to convert measuring radiation emitted by the radiation source into an electrical measuring signal and to output to the control unit 33.

The analysis device 1 further includes a fluid conduit 14 for conveying a liquid sample from a sample template not shown in detail. The liquid line 14 for the liquid sample is connected to the mixing cell 31 via a further fluid conduit 16. The control unit 33 conveys the liquid sample to pretreatment by means of a corresponding control of the pump drive 36 and the central control station 34 by means of the actuator M for actuating the valve V1 in the mixing cell 31.

The measurement variable to be determined by the analyzer is a measurement parameter correlated to the concentration of one or more substances in the liquid sample. The analyzer 1 comprises three liquid containers 9, 11, and 13 that contain reagents, which are simultaneously or successively mixed with the liquid sample in order to effect a detectable change in the fluid sample, for example, a color or a color change via the photometric measuring device 35, by means of a chemical reaction with one or several substances that are part of the measurement parameters. The control unit 33 can meter the reagents 42 into the mixing cell 31 where it is mixed with the liquid sample by operating the pump drive 40, 41, 42 with the valves V5, V6 and V7. For photometrically determining the measured variable, the reaction mixture produced by mixing the fluid sample with the reagent can be further transported to the measuring cell 32. The measuring cell 32 includes optical windows which are transparent to measured radiation emitted by the radiation source 52. The measured radiation transmitted from the reaction mixture measured is received by the radiation receiver 52, which outputs a measurement signal correlated to the intensity of the transmitted radiation to the control unit 33. This is designed to derive a measurement value from the measurement signal for the measurement variable that is to be determined, and to store it, and/or output it via a user interface or to a parent unit.

The analyzer 1 has three additional reservoirs 3, 5 and 7. The reservoir 3, which is connected to the mixing cell 31 via the same liquid line 16 as the fluid line 14, via which the liquid sample is taken from the sample template, t contains a cleaning solution. The two additional storage containers 4 and 5 each contain a standard solution. The standard solution contained in the reservoir 4 can have a known value of the measurement variable that is to be determined. The standard solution contained in the reservoir 5 may contain distilled water serving as a zero standard. By means of piston pumps S1, S2 and S3 and in conjunction with the valves V1, V2 and V3, these liquids can be controlled by the control unit via the liquid lines 15, 16, 17, 18, 19 and 20 to the mixing cell 31 and further conveyed to the measuring cell 32.

The mixing cell 31 is connected to a waste container that is not shown via a fluid path passing through the fluid lines 23 and 21, and the fluid path can be blocked by means of the valve V4. The pump S4 serves to transport the liquid from the measuring cell 32 through the mixing cell 31 and the fluid lines 23 and 21 to the waste container.

The liquid containers 3, 5, 7, 9, 11, 13, parts of piston pumps S1, S2, S3, S4, S5, S6, S7, at least a portion of the system of fluid lines and the mixing cell 31 may be arranged in a replaceable cassette which can be connected to and disconnected from an analyzer base structure. The analyzer base structure comprises the other components of the analyzer 1, for example, the measuring cell 32, the measuring device 35, the control unit 33, the pump drives 36, 37, 38, 39, 40, 41, 42 for the piston pumps S1, S2, S3, S4, S5, S6, S7, the drive for the central M switching mechanism 34, and optionally, other components, such as a ventilation and/or cooling system for the analyzer. The analyzer base structure can be formed as one piece or be comprised of several, especially interconnected modules that can be connected to and disconnected from each other.

The analyzer 1 is configured to allow a user to select the measurement variable that is to be determined from a plurality of possible measurement parameters. For this purpose, the control unit 33 has a data memory where a computer program is stored. In the computer program, at least one measurement-parameter-specific control algorithm and/or at least one measurement-parameter-specific evaluation algorithm is implemented for each of the possible measurement parameters. In an alternative embodiment, the control unit 33 can access a remote data memory unit in which a computer program is stored, accessing it via a communication link, such as a network connection or via a field bus.

Using a selection signal representing one measurement variable of the plurality of measurement parameters, which the user is able to generate by entering an input through the input device of the control unit 33, the control unit 33 is signaled that, in order to control the analyzer 1 and in order to evaluate the measurement signals provided by the measurement device 35, only those control and evaluation algorithms used for determining the selected measurement variable shall be executed. It is not excluded that non-measurement-parameter-specific, overarching control algorithms that can be used to determine several of the selectable measurement parameters, e.g. a control algorithm for conveying sample fluid from the sample template or for conveying cleaning liquid from the liquid container 3 through the measuring cell 32, are used in the context of the determining the selected measurement variable. Excluded, however, is the application of measurement-parameter-specific control or evaluation algorithms that are used in determining a measurement parameter other than the selected measurement variable, and which are not universally applicable in order to determine several or all of the parameters.

The computing device of the control unit 33 may include a processor with an internal memory in which an executable basic program is included that the processor can execute, and which provides certain basic functions of the control unit 33. In particular, this basic program may be configured to detect the selection signal, and, based on the selection signal, to install the computer program modules—which comprise how the control and evaluation algorithms are used to determine the selected measurement variable—in the internal memory of the processor, to which the processor has access so that it can control the analyzer. It is therefore not necessary that all measurement-parameter-specific control and evaluation algorithms be stored in the processor's limited internal memory.

A measurement-parameter-specific control algorithm, for example, comprises a predetermined sequence of pumping and metering of reagents from the containers 9, 11 and 13. If determining a measurement parameter by adding more or fewer reagents, then the number of containers and the number of conveying and metering steps—in particular, the control of the pumps and of the central valve switching station 34—required to form the reaction mixture from the reagents and the liquid sample are adjusted according to the measurement parameter. Similarly, a measurement-parameter-specific control algorithm can comprise the control for the radiation source 51 in such a way that it emits radiation that has a wavelength which is suitable for determining the respective measurement parameter. In the present example, in which the radiation source 51 comprises a plurality of LEDs, a measurement-parameter-specific control of the radiation source provides the excitation of one or more LEDs, which emit correspondingly suitable radiation as measurement radiation. In implementing such a measurement-parameter-specific control algorithm, the control unit 33 actuates the pumps S1, S2, S3, S4, S5, S6, S7, and the central valve switching device 34 in a predetermined manner via the control algorithm.

A measurement-parameter-specific evaluation algorithm includes instructions by which the specific measurement parameter is derived from the measurement signal, which is provided by the radiation receiver 52.

If a user selects a measurement variable from the plurality of possible measurement parameters, then the user is providing the proper reagents, standard solutions and cleaning fluids required for determining the selected measurement variable in the liquids containers 3, 5, 7, 9, 11 and 13.

The sequence of an analytical method for determining a measurement variable selected from a plurality of possible measurement parameters shall be described below with reference to FIG. 1.

At the beginning of an analysis cycle, the liquid sample is transported along an initial fluid path along the fluid lines 14 and 16 into the mixing cell 31. The valve position of the valve V1 is controlled by the central valve-switching mechanism 34 via the control unit 33 in such a way that an initial fluids path section from the sample template to the piston pump S1 is opened, while a second fluids path section is blocked from the piston pump S1 to the mixing cell 31. Another fluid path running from the liquid container 3, which contains a cleaning fluid, to the piston pump is blocked in this valve position on the liquid line 15. After one of the liquid quantities, which is predetermined by the control unit 33 via appropriate control on the part of the pump drive 36, has been fed into the piston pump S1, the valve V1 is switched to a second valve position wherein the second fluid path section is opened, while the first fluid path section is blocked, and the fluid path to the liquid container containing cleaning fluid 3 is also disabled. The control unit then triggers the pump drive 36 in a suitable manner in order to meter a predetermined amount of the liquid sample into the mixing cell 31. At the same time or in succession, reagents from the liquid containers 9, 11 and 13 can be metered into the mixing cell 31. For this purpose, the valves V5, V6 and V7 are switched to an initial valve position in which the respective first fluid path section leading from the fluid containers 9, 11 and 13 via the liquid conduits 24, 26 and 28 into the cylinders of the piston pumps, S5, S6 and S7 is opened, whereas the second fluid path section leading from the piston pumps 55, S6 and S7 via the fluid lines 25, 27 and 29 in the mixing cell 31 is opened. In this position, the piston pumps 55, S6, S7 may each aspirate an amount of liquid into their cylinders that the control unit 33 predetermines. Thereafter, the valves V5, V6 and V7 are switched to a second valve position in which the second fluid path section is opened and the first fluid path section is closed. The control unit is able to then actuate the pump drive 40, 41 and 42 to meter the desired volume of reagent into the mixing cell 31. Since an individual piston pump with an individual pump drive is associated with each reagent, then each reagent can be transported into the mixing cell 31 at a different flow rate and/or variable volume.

In the mixing cell 31, the reagents are mixed with the liquid sample to form a reaction mixture. In this case, a chemical reaction takes place with one or more substances which influence the measured variable, which leads to a change that is detectable photometrically. The reaction mixture continues to be transported into the measuring cell 32 by means of the piston pump S1, where the photometric measuring device 35 detects a measurement signal that is a function of the measurement variable that is to be determined. The control unit 33 receives the measuring signal of the photometric measuring means 35 and from that derives the concentration of analyte present in the fluid sample. After the measurement has been completed, then, by means of the valve V4, the measuring cell 32 is discharged by opening a fluid path from the measurement cell 32 via the mixing cell 31 and the liquid conduit 23 into the piston pump S4, and the piston pump S4 is actuated in order to suction off the spent reaction mixture from the measurement cell 32 and transport it to a waste container. The analytical cycle is then complete.

The analysis cycle can also include in addition a zero-line measurement in which, in a manner similar to the measurement described earlier, the piston pump S1 conveys a liquid sample into the measuring cell 32 without adding reagents from the containers 9, 11 or 13. Using sensor 35, a zero signal can be determined on the fluid sample contained in the measurement cell whose value is stored by the control unit 33, and which is taken into consideration in determining the measurement variable of a reaction mixture formed by mixing a liquid sample with the reagents.

The analyzer 1 can repeatedly perform such an analysis cycle. In addition, between the analysis cycles or after carrying out a number of analysis cycles, a cleaning step may also be performed in which, by the means of the piston pump S1, which, in the example shown here, also serves to convey the liquid sample into the mixing cell 31, the piston pump S1 pumps a cleaning liquid out of the container 3 into the mixing cell 31 and then conveys it on to the measuring cell 32, where the piston pump S4 resuctions it and discharges the cleaning liquid into a waste container. Liquid transport takes place, as described in detail by way of the measurement cycle, by operating the pump drives 36 and 39 and by appropriate switching of the valves V1 and V4 by means of the valve central switching station 34.

At predetermined intervals, for example after a predetermined number of analysis cycles, or prior to execution of each analysis cycle, one or more calibration measurements can be carried out, during which, instead of conveying a liquid sample from the sample template, a standard solution is conveyed from the liquid containers 5 and/or 7 by means of piston pumps S2 and/or S3, and by the associated valves V2 and V3, which are actuated by the central switching device in a manner similar to the one described above for feeding the reagents from the containers 9, 11 and 13 into the mixing cell 31. The flow of a calibration measurement is carried out, moreover, in the same way as an analysis cycle with a liquid sample from the sample template.

For (permanent) conversion of the analyzer 1 from being used to determine the selected measurement variable that was first selected to determining a second, different measurement variable that is different from the first measured variable, e.g. ammonium content being the first measurement variable and phosphate content being the second measurement variable, the user can replace the liquid containers 9, 11, 13, 5 and 7, which contain the reagents and standard solutions for the determination of the first selected measured variable, with a liquid containers containing reagents and standard solutions that are correspondingly used for determining the second measured variable. As described in detail above in 10 2009 029 305 A1 and DE 10 2011 075 762 A1, the liquid containers may be integrated in a replaceable cartridge.

Simultaneously, the user can input the new, second measurement variable selected from said plurality of measurement parameters by means of the inputting unit that is part of the control unit 33. Alternatively, a “plug-and-play” operation can be achieved by connecting one or more of the liquid containers to a memory module from which the control unit can read—e.g. via its interface that was mentioned earlier that can be connected to the control unit 33, which can be connected to an external memory storage unit—the measurement variable selected as a selection signal that is used for identification purposes. In this embodiment, the user does not have to input anything on the control unit 33.

Based on the selection signal, the control unit 33 identifies the measurement-parameter-specific control and evaluation algorithms that have to subsequently be executed to determine the newly selected second parameter. As described above, this can be carried out in such manner that a computer processor for the control unit 33 installs computer program modules in an internal memory unit of the processor by means of a basic program to which the processor has access, and these computer modules comprise control and evaluation algorithms used in determining the selected second measurement variables so that this computer processor can run these modules as described below. Next, operation of the analysis device 1 can be continued, wherein the analysis device 1 now provides measurement values for the new, second selected measurement variable.

It is also possible for the analyzer 1 to temporarily switch from a first selected measurement variable to determining a second selected measurement variable that is different from the first selected measurement variable, and then return to the first measurement variable. Since in this case no permanent conversion is desired, it is advantageous to put the reagents and standard solutions that are used to determine the first and second selected measurement variable in liquid containers 9, 11, 13, 5, and 7 simultaneously. In this case, the control unit 33 is designed to control the conveying and metering device with the valves V1, V2, V3, V4, V5, V6 and V7, the piston pumps S1, S2, S3, S4, S5, S6 and S7, and the central valve switching mechanism 34 in such a way that, depending on the currently selected measurement variable, the corresponding reagents used for determining the currently selected measurement variable are conveyed in the proper sequence and are added to the liquid sample.

The control unit 33 or another computing device connected to the control unit 33 for communications purposes can be configured to run a computer program that is used to generate time or event-oriented selection signals that represent the first or the second selected measurement variable. Based on these selection signals, the control unit 33 then controls a conveying and metering device according to a measurement-parameter-specific control algorithm used for determining the first or second selected measurement variable, or it determines the current measurement value for each selected measurement variable according to a measurement-parameter-specific evaluation algorithm.

Temporarily switching the analysis instrument back and forth between a first and a second selected measurement variable can be advantageously used for monitoring phosphate removal processes in sewage treatment processes. In this situation, the control unit 33 can switch back and forth between an initial “phosphate-yellow” measurement variable, i.e. the one with the phosphate content that the vanadate molybdate method (yellow method) is able to detect, and a second “phosphate-blue” measurement variable, i.e. the phosphate content in the liquid sample, that the molybdenum blue method is able to detect. Whereas a wavelength of 380 nm serves as a peak wavelength for the radiation source in the vanadate molybdate method 51, a peak wavelength of 880 nm is used in the molybdenum blue method. Depending on the nature of the liquid sample, e.g. its turbidity or color, the one or the other measurement variable provides more accurate results. In this situation, event-driven switching is advantageous in response to characteristics in the liquid sample. For example, it is possible that the control unit can determine a property in the monitored liquid, and can do this based on a calibration measurement or signal from a turbidity sensor that can be added, and can generate a selection signal based on this property, which more reliably represents the phosphate-yellow and phosphate-blue measurement variables for the nature of each liquid.

1. An analyzer for automatically determining a measurement variable of a liquid sample, comprising:
at least one liquid container for a reagent that is to be added to the liquid sample;
a measuring cell for receiving a reaction mixture formed by the liquid sample mixed with the reagent;
a photometric measuring system for providing a measurement signal correlated with the measurement variable for the reaction mixture received in the measuring cell;
a conveying and metering device for conveying and metering the liquid sample and reagent into the measuring cell; and
an electronic control unit operative to perform the following functions:
(a) receive a selection signal representing a measurement variable selected from a plurality of measurement parameters; and
(b) automatically determine the measurement variable based on the selection signal by:
controlling at least the conveying and metering device and the photometric measuring system, and/or
evaluating the measurement signal received from the photometric measuring system.

2. The analyzer according to claim 1, wherein each of the plurality of measurement parameters correlates to a concentration of one or more substances in the liquid sample.

3. The analyzer according to claim 1, further including a memory for storing a plurality of measurement-parameter-specific algorithms, including one or more measurement-specific algorithms associated with each one of the measurement parameters;
wherein the electronic control unit is operative to execute, based on the selection signal, those measurement-parameter-specific algorithms that serve to determine the selected measurement variable; and
wherein the measurement-parameter specific algorithms are either:
(a) control algorithms serving to control the analyzer to determine the corresponding measurement parameter, or
(b) evaluating algorithms serving to evaluate a measured value of the measurement signal to determine a measured value of the corresponding measurement parameter.

4. The analyzer according to claim 2, further including a memory for storing a plurality of measurement-parameter-specific algorithms, including one or more measurement-specific algorithms associated with each one of the measurement parameters;
wherein the electronic control unit is operative to execute, based on the selection signal, those measurement-parameter-specific algorithms that serve to determine the selected measurement variable; and
wherein the measurement-parameter specific algorithms are either:
(a) control algorithms serving to control the analyzer to determine the corresponding measurement parameter, or
(b) evaluating algorithms serving to evaluate a measured value of the measurement signal to determine a measured value of the corresponding measurement parameter.

5. The analyzer according to claim 1, wherein the control unit includes an input for selecting the selected measurement variable and for generating the selection signal that represents the selected measurement variable based on the selection.

6. The analyzer according to claim 1, wherein the control unit is configured to read out the selection signal from a data storage memory that can be connected to, and disconnected from, the control unit.

7. The analyzer according to claim 1, wherein the photometric measurement device comprises a radiation source and a radiation receiver that outputs a measurement signal dependent on the intensity of radiation received;
wherein the radiation source and the radiation receiver are configured in such a way that the radiation emitted by the radiation source flows along a measurement path through the reaction mixture contained in the measurement cell; and
wherein the wavelength of the radiation source is selectable by the control unit.

8. The analyzer according to claim 1, wherein the radiation source comprises a plurality of light-emitting diodes or multi-LEDs, each configured to produce radiation with a separate and discrete peak wavelength selectable by the control unit.

9. The analyzer according to claim 8, wherein the control unit is operative to control one of the plurality of light-emitting diodes or multi-LEDs to emit radiation serving to determine the selected measurement variable.

10. A method for determining a measurement variable, comprising the steps of:
providing the analyzer of claim 1;
providing one or more of the liquid containers with reagents used in forming a reaction mixture with a fluid sample;
selecting a measurement variable from a plurality of the measurement parameters through an input device in communication with the control unit;
conveying and metering a liquid sample from a sample preparation;
conveying and metering one or more reagents from one or more fluid reservoirs with the liquid sample to form a reaction mixture;
conveying and metering the reaction mixture to the measuring cell;
controlling a photometric measuring system associated with the measuring cell to produce a measurement signal dependent on the selected measurement variable; and
determining a measurement value for the selected measurement variable based on the measurement signal.

11. The method according to claim 10, wherein, based on the selection of the selected measurement variable, the control unit executes a measurement-parameter-specific algorithm to control at least the conveying and metering device and the photometric measurement device for evaluating the measurement signal provided by the photometric measurement device.

12. The method according to claim 10, further including the steps of:
controlling a radiation source of the photometric measurement device to generate a measurement radiation traveling through a reaction mixture received in the measurement cell, wherein the measurement radiation has a wavelength selected based on the selection signal representing the selected measurement variable; and
detecting a measurement signal produced by a radiation receiver of the photometric measuring system, wherein the measurement signal is dependent on an intensity of the measuring radiation striking the radiation receiver after traveling through the reaction mixture.

13. The method according to claim 11, further including the steps of:
controlling a radiation source of the photometric measurement device to generate a measurement radiation traveling through a reaction mixture received in the measurement cell, wherein the measurement radiation has a wavelength based on the selection signal representing the selected measurement variable; and
detecting a measurement signal produced by a radiation receiver of the photometric measuring system, wherein the measurement signal is dependent on an intensity of the measuring radiation striking the radiation receiver after traveling through the reaction mixture.
